(19) United States
(12) Patent Application Publication
(10) Pub. No.: US 2017/0197026 A1
(43) Pub. Date: Jul. 13, 2017

(54) METHODS CIRCUITS DEVICES ASSEMBLIES SYSTEMS AND FUNCTIONALLY ASSOCIATED COMPUTER EXECUTABLE CODE FOR TRANSPORTING OR PROVIDING FLUID TO A PATIENT

(71) Applicant: Q-CORE MEDICAL LTD., Netanya (IL)

(72) Inventors: Dafna Kesselman, Tel Aviv (IL); Shaul Eitan, Hofit (IL); Andrei Yosef, Even Yehuda (IL)

(21) Appl. No.: 15/401,122

(22) Filed: Jan. 9, 2017

Related U.S. Application Data
(60) Provisional application No. 62/276,876, filed on Jan. 10, 2016.

(51) Int. Cl. A61M 5/142; G06F 19/00; A61J 15/00

(52) U.S. Cl. CPC A61M 5/142; A61J 15/0015; G06F 19/3468; A61M 2205/502; A61M 2205/52; A61M 2205/3576; A61M 2005/14208

(57) ABSTRACT

Disclosed is a medical device including a pumping mechanism for administering a fluid to a patient, where the pumping mechanism is characterized as having two or more treatment routes by which to connect to a patient, a controller for controlling the pumping mechanism, one or more inputs for receiving information including an intended route indication, and a display to visually label the medical device according to the selected route indication.

### RELATED APPLICATIONS

The present application claims priority from U.S. Provisional Patent Application No. 62/276,876, entitled: “METHODS CIRCUITS DEVICES ASSEMBLIES SYSTEMS AND FUNCTIONALLY ASSOCIATED COMPUTER EXECUTABLE CODE FOR TRANSPORTING OR PROVIDING FLUID TO A PATIENT”, filed on Jan. 10, 2016 which is hereby incorporated by reference into the present application in its entirety

### FIELD OF THE INVENTION

The present invention relates generally to the field of medical devices for providing fluid to a patient. More specifically, the present invention relates to operating a medical device at least partially based on route information.

### BACKGROUND

Medical devices operate for therapeutic and/or diagnostic uses. Some exemplary medical devices may be: blood pressure monitors which may monitor a patient's blood pressure and heart rate, electrical thermometers which may measure a patient's body temperature and many more.

Some medical devices may administer fluid to a patient via a conduit such as a flexible tube or a cassette including at least some flexible segments and some fixed segments. Some medical devices may monitor fluid flowing through its system and connected to one or more of a patient's bodily fluids. For example: an infusion pump which may be used to infuse fluids into a patient. In another example, a dialysis machine may pass a patient's blood through the machine to filter and get rid of toxins and excess fluids. Some medical devices may be configured to operate on a specific part of a patient's body while others are configured to operate or connect to a plurality of parts. Some medical devices for administering fluid, may connect to a plurality of routes such as intravenous, subcutaneous and more.

A medical device may be used in a hospital, doctor or nurse's office or other medical treatment centers. Medical devices may also be used at patient's homes or personal environments.

### SUMMARY OF THE INVENTION

The present invention includes methods circuits devices assemblies systems and functionally associated computer executable code for transporting or providing fluid to a patient. According to some embodiments, a medical device may include a pumping mechanism for administering a fluid to a patient, where the pumping mechanism is characterized as having two or more treatment routes by which to connect to a patient, a controller for controlling the pumping mechanism, one or more inputs for receiving information including an intended route indication, and a display to visually label the medical device according to the selected route indication. An intended route indication may be: intra-venous (IV), subcutaneous, epidural, enteral, intrathecal, subarachnoid, peri-neural, neuro-axial and more. A pump mechanism for administering fluid may be: saline, water, total parenteral nutrition (TPN), lipids, IV medication, epidural medication, blood, blood products or otherwise.

According to some embodiments, a medical device may include a memory storing a drug library including permissible route information. A controller may compare an intended route indication with permissible route information and disable the pumping mechanism if an unallowable (or non-matched or incompatible) combination is indicated. The controller may be configured to compare the intended route indication and disable the pumping mechanism if the actual treatment route and the intended treatment route are non-matched or incompatible. Visually labeling a medical device may include: coloring a screen background, enabling a LED emitting a specific color, displaying a route name or otherwise. The display may also display a ward in which the medical device is operating.

According to some embodiments, a medical server may include: a drug library including a permissible route field; program setting to be relayed to a medical device including a medical device identification and an intended treatment; and a transceiver to (a) periodically relay to one or more medical devices the drug library to be stored locally on the one or more medical device and to (b) relay to a specific medical device of the one or more medical devices a program setting, the specific medical device may compare the program setting with the permissible routes of the stored drug library and to disable the medical device if they are non-matched.

According to some embodiments, a method of operating a medical device may include: selecting a treatment route, correlating between the treatment route with one or more program parameters; producing an indication indicating either a non-allowable combination if the treatment route is incompatible with said at least one or more parameters or an allowable combination; and disabling the medical device if the indication is a non-allowable combination. The method may include visually displaying said treatment route. The program parameters may include: a fluid type, a delivery mode, a dose of the fluid, the time of the treatment, and an identification of the medical device. The method may include receiving a drug library including at least one permissible route for each entry in the drug library and/or visually displaying a ward where the medical device is located.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows a medical device environment in accordance with some embodiments;

FIG. 2 shows a medical device system in accordance with some embodiments; and

FIG. 3 shows a flow chart for operating a medical device.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, components and circuits have not been described in detail so as not to obscure the present invention.

Unless specifically stated otherwise, as apparent from the following discussions, it is appreciated that throughout the specification discussions utilizing terms such as “processing”, “computing”, “calculating”, “determining”, or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Embodiments of the present invention may include apparatuses for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

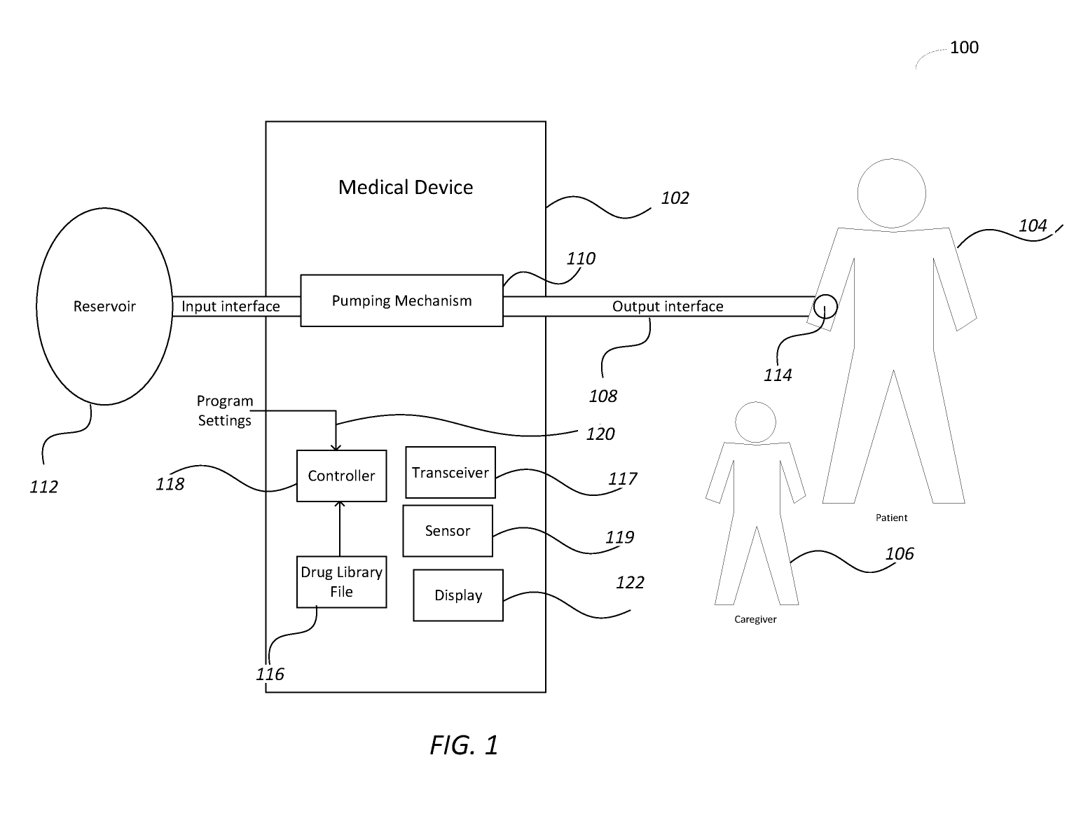

Turning to FIG. 1, shown is a medical device environment 100 in accordance with some embodiments, including a medical device such as medical device 102 and a subject or patient such as patient 104. Optionally there may also be a caregiver such as caregiver 106. It is understood that patient 104 and caregiver 106 may be the same person. Alternatively, caregiver 106 may be a nurse, doctor, pharmacist, family member or otherwise.

According to some embodiments, medical device 102 may provide a treatment or fluids to patient 104. Medical device 102 may include a pumping device/mechanism such as pumping mechanism 110. Pumping mechanism 110 may include a multi-mode motorized fluid pumping assembly. Pumping mechanism 110 may be configured to receive fluid through an input interface (such as a conduit) from an infusible fluid source such as reservoir 112, (which may be an infusion bag or otherwise), and to expel or otherwise provide fluid to patient 104 via an output interface 108 (which may be a conduit or otherwise). Output interface 108 may further include a delivery set. Output interface 108 may be configured to connect/include with each of a variety of different infusion delivery set types, and may also be configured to deliver an infusion to a different part of a subject's body as depicted by route 114. Route 114 may be a destination or part of a subject's body to which fluid is intended to be delivered. Some example routes may include: Intra-venous (IV), subcutaneous, epidural, enteral, intrathecal, subarachnoid, peri-neural, neuro-axial and more. Accordingly, output interface 108 may be selected from different delivery set types which may be used to deliver the fluid to different parts of patient 104's body. The fluid may be saline, water, Total Parenteral Nutrition (TPN), lipids, IV medication, epidural medication, blood and blood products or a combination of any of these and more.

According to some embodiments, medical device 102 may include a drug library source such as drug library 116. Drug library 116 may be stored in a dedicated memory or received via transceiver 117 and stored in the controller 118. Controller 118 may be configured to control pumping mechanism 110.

According to some embodiments, drug library 116 may be updated periodically, while medical device 102 is inactive and/or while pumping mechanism 110 is not administering fluid to patient 104. Optionally, the drug library may be received via transceiver 117 while pumping mechanism 110 is active but may only be updated while pumping mechanism 110 is inactive or medical device 102 is idle. Drug library 116 may include a table or list of information which includes and correlates information such as (a) a fluid type, (b) delivery mode (c) the route to be used (d) the dose of the fluid if applicable and more. A drug library file/database 116 may be produced and/or controlled by a pharmacist or medical professional to define allowable drug/fluid administration options. A drug library database may enable caregivers to select a prescription/administration selection/drug library entry out of a database of allowable administration/prescription options. So that a caregiver (such as a physician, nurse, pharmacist or otherwise authorized personnel) may select a requested prescription out of the database thus increasing safety and lowering mistakes in prescribing a drug.

According to some embodiments, some example delivery modes may include continuous, multi-step, secondary, PCA, Taper, intermittent, bolus, and/or loading dose, or any combination of these.

According to some embodiments, a drug library database may include a route field or a drug library vector. In a route field embodiment, a caregiver may select a drug to be administered, a drug concentration/amount, a drug administration rate and/or a delivery mode and the available routes applicable to the selected fields may then be selectable to a user. In a vector configuration a caregiver may select a vector/drug library entry which includes all of the applicable components together (such as drug type, route and delivery mode etc.). So for example, in a field embodiment a caregiver might select drug A, at concentration B mg per liter and then might be able to select a route (such as epidural and/or intravenous) for A and B. In the same example a caregiver could select either vector 1 (A, B, route epidural) or vector 2 (A, B, route intravenous). Optionally, the route information may be a group of allowable routes and not a single route.

According to some embodiments, medical device 102 may be configured to receive program settings 120, which optionally may be relayed to controller 118. Program settings 120 may either be entered/input locally by caregiver 106 or may be received via transceiver 117 from a remote server. Optionally, if program settings 120 are received from a remote source the input details may be confirmed locally by caregiver 106.

According to some embodiments, controller 118 may be configured to check if program settings 120 is allowable compared to an appropriate drug library element of drug library 116. Accordingly, controller 118 may correlate at least two of the following intended parameters (which may be received at program settings 120 and drug library 116): ((a) the fluid type, (b) the mode of pump assembly operation/delivery mode (c) intended route to be used (d) the dose of the fluid if applicable; (e) the time of the treatment, and/or (f) medical device 102 identification. At least one of these intended parameters may be received at program settings 120 and compared with an element of drug library file 116. Furthermore, controller 118 may compare one of the intended parameters with an actual parameter, either by confirming a separate input scanned or input by a caregiver (that the actual reservoir 112 is filled with the expected fluid type) or automatically by utilizing a detector of medical device 102. If compared parameters do not match then the controller may indicate that they are non-matched. It is noted that while program settings 120 may include two or more parameters, where program settings 120 is discussed in this application it is understood that one or more of the parameters of program settings 120 is intended.

Some examples may include: controller 118 may confirm that an intended route is allowable based on drug library file 116. In a further example, controller 118 may also confirm that the actual route 114 is the intended and allowable route. For example if the selected route is epidural if medical device output interface 108 is connected intravenously it may be automatically detected. Controller 118 may compare the expected parameters with allowed parameters or ranges of parameters.

According to some embodiments, controller 118 may be configured to correlate a mode of operation of the medical device 102 and type of a delivery set connected to output interface 108.

According to some embodiments, if the comparison of an intended state based on the program settings 120 with drug library file 116 and/or the actual state of the medical device (actual reservoir fluid, actual route etc.) indicates at least one unallowable combination (i.e. that two or more parameters are non-matched) then controller 118 may cause pumping mechanism 110 to stop or to be at least partially disabled to increase patient 104's safety.

According to some embodiments medical device 102 may include a display such as display 122. Display 122 may be configured to audibly and/or visually relay information to caregiver 106 and/or patient 104. For example, if medical device 102 disables pumping mechanism 110 as discussed in an example above, then display 122 may emit an audible alarm and/or a visual alarm and may also display relevant information, such as which intended parameters did not match which actual or allowable parameter(s).

According to some embodiments, display 122 may be configured to visually display the intended route, which may be received from drug library 116 and/or from program settings 120. Clearly displaying the intended route may aid caregiver 106 in caring for a patient as in some treatment surroundings a plurality of medical devices 102 may be active near each other, optionally on the same patient 104 and being able to quickly understand which route a medical device 102 is intended for may improve caregiver 106 user experience of medical device 102 and improve safety of operation of medical device 102.

According to some embodiments, display 122 may also indicate the clinical care area (CCA) that medical device 102 is associated with. For example, at times caregiver 106 may select a “General file” out of drug library 116 which does not designate a specific element out of the list. So that displaying the CCA that a medical device may be associated with such as intensive care unit (ICU) may be advantageous to warn a subsequent caregiver such as caregiver 106 that medical device 102 was programmed in a specific CCA and if patient 104 was moved to a different CCA they may be more aware of the CCA medical device 102 was designated in. This may be advantageous as patient 104 may be moved mid treatment between CCAs and may aid in treating the patient 104 safely and efficiently.

According to some embodiments, display 122 may visually indicate a route and/or a ward by having one or more LEDS or other light emitting elements emit a specific color and/or pattern or combination of colors. Alternatively, a specific light associated with a route may be turned on to indicate that route has been selected. Optionally, the background of a display may be a specific color or pattern to indicate a selected route and/or ward. Optionally, the route and/or ward may be written on the display.

According to some embodiments, program settings 120 and/or actual parameters may be input into medical device 102 using an input device such as a touch screen or barcode reader, camera or otherwise. Accordingly, medical device 102 may include an input device such as a touch screen, barcode reader, camera, sensor and more. For example, an actual route 114 may be input to the medical device using a scanning device to scan a barcode near the route 114.

According to further embodiments, controller 118 may enforce route compliance by requiring caregiver 106 to confirm attachment, to the correct destination at the patient 104's body. According to further embodiments, controller 118 may enforce route compliance by requiring caregiver 106 to confirm attachment of a delivery set of output interface 108 of a type indicated as acceptable by controller 118. According to yet further embodiments, output interface 108 and/or another portion of medical device 102 may include and/or be otherwise functionally associated with a compliance sensor 119 adapted to detect which type of delivery set is connected to the output interface 108, and optionally signal controller 118 to block pumping mechanism 110 from operation if the type of the connected set is not a permissible type/route for the mode of operation. Compliance sensor 119 may be an optical sensor, such as a bar code scanner or an image acquisition assembly (e.g. CMOS sensor module) configured to read a bar code or other visible feature of the delivery set of output interface 108. Compliance sensor 119 may be a radio frequency sensor, such as an RFID scanner configured to read an RFID tag attached to the delivery set or otherwise.

According to further embodiments, based on an indication of which type of fluid is connected to the input interface of the pump, controller 118 may indicate, through display 122 which may include speakers, which modes of operation may be selected by caregiver 106 and employed by the motorized pumping assembly of pumping mechanism 110. Medical device 102 may include or be communicatively coupled with drug library 116 which may correlate drug types with permissible delivery modes and further correlates drug and mode combinations with permissible routes per combination and additional medicinal information and data associated with the optional treatments and fluid types. According to some embodiments, medical device 102 may compare the received parameters and may determine if two or more parameters are compatible and/or correlated or non-correlated/incompatible or non-matched. The parameters may be received from one or more remote servers, user input, scanned information (RF ID, barcode, Qcode and more) or may be detected by one or more compliance sensor(s) 119 or a combination of these and more.

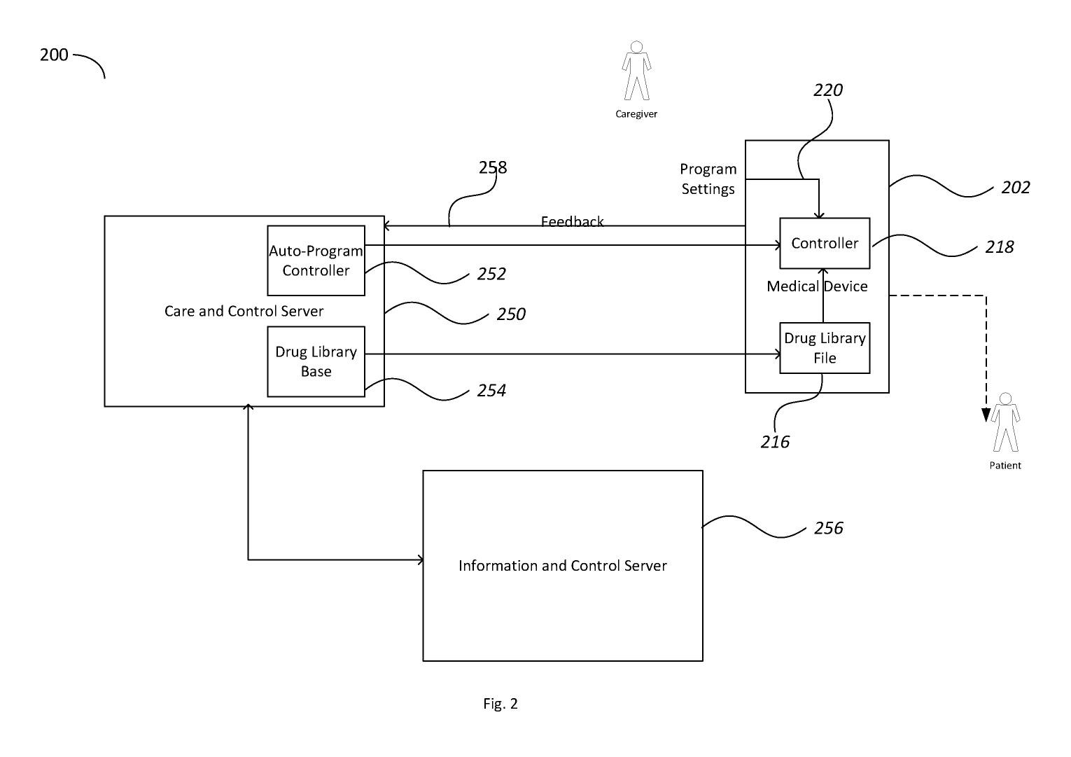

Turning to FIG. 2 depicted is a medical device system 200. According to some embodiments, system 200 may include a medical device 202 which is substantially similar to medical device 102 of FIG. 1. Appropriately controller 218, drug library file 216 and/or program settings 220 are substantially similar to the controller 118, drug library file 116 and/or program settings 120 of FIG. 1. System 200 may also include a care and control server 250 configured to communicate with medical device 202. Care and control server 250 may be configured to relay to medical device 202 information such as, a requested treatment for a user, medical device updates, drug library updates from a drug library based controller 254 and more. Optionally, medical device 202 may be programmed by program setting input 220 and/or auto program controller 252 and/or a combination of the two.

According to some embodiments, System 200 may include an information and control server 256 which may be a local server configured to store and manage local patient files and drug library and may further be configured to relay local information (such as the drug library) to one or more medical devices such as medical device 202 via care and control server 250. Information and control server 256 may receive information from medical device 202 and/or via medical device feedback 250 via care and control server 250. Information and control server 256 includes patient information so that a comparison of patient information may be compared to drug library information at this point and an incompatible indication may be related to the medical device if a non-matched comparison is detected. For example, if a drug library selection incompatible with a patient's weight is selected, or alternatively if a patient is allergic to the drug of the drug library selection.

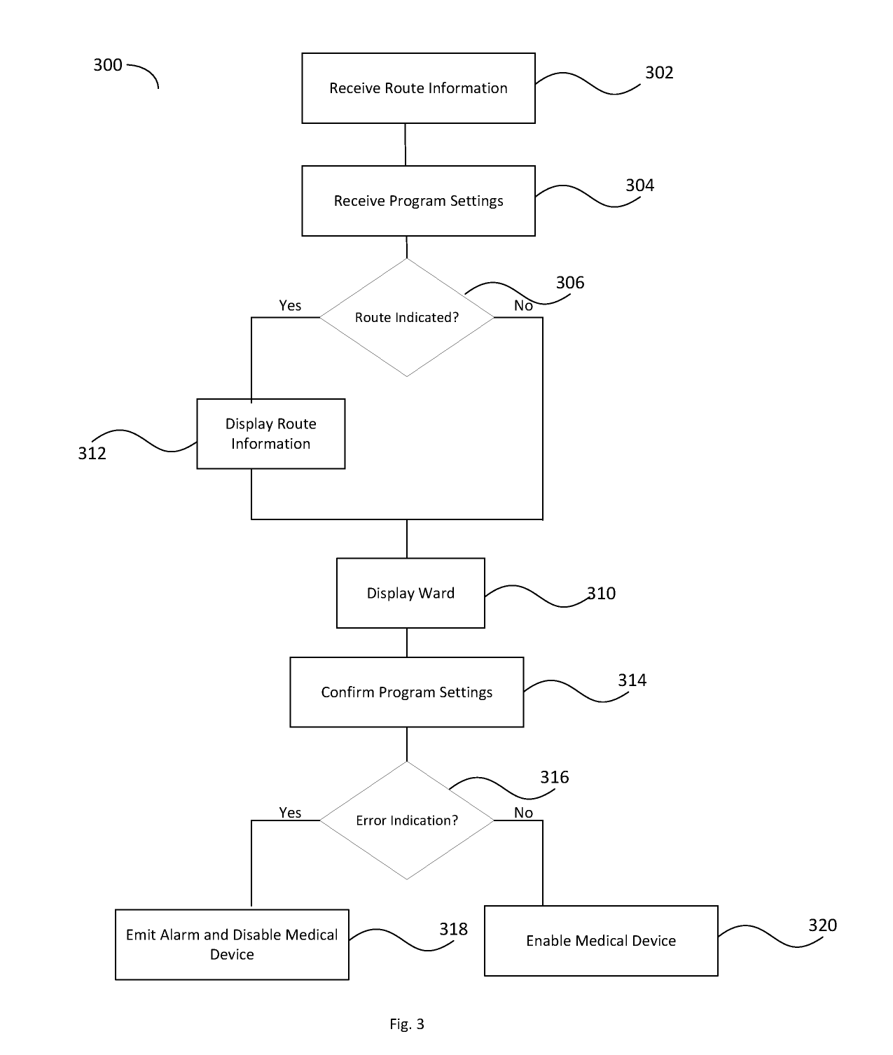

Turning to FIG. 3 shown is a method for operating a medical device 300. A medical device may receive a program setting (step 304) and route information (step 302). The program setting may be inserted manually by a user or may be programmed remotely. The route information may be received within the program setting and/or remotely or locally from a drug library element or otherwise. If a route is indicated (step 306) then the route may be audibly or visually displayed (step 312). Optionally a ward associated with the treatment or where the treatment is being administered is displayed (step 310). A ward may also be displayed in conjunction with route information.

According to some embodiments, the received program settings may be confirmed locally either automatically or by a user or semi-automatically using a scanning device (step 314). For example, a caregiver may scan associated barcodes on each of the associated parts and confirm the program settings are correct (route is the intended route etc.). Optionally, some of the program settings may be detected automatically using a compliance sensor of the medical device, for example, a pressure sensor may be able to differentiate between different types of output interfaces connected.

Furthermore, confirmation of program settings may include confirming that all of the settings are permissible/allowable. For example that an actual route is allowable with the intended drug and more. Checking allowability may include comparing intended programming settings with a drug library and/or a combination of the two.

According to some embodiments, if one or more of the intended program setting are not the actual settings or are not allowable (step 316), then the medical device may emit an alarm and/or disable the medical device (step 318). If no error is indicated then the medical device may be enabled (step 320).

While certain features of the invention have been illustrated and described herein, many modifications, substitutions, changes, and equivalents will now occur to those skilled in the art. It is, therefore, to be understood that the appended claims are intended to cover all such modifications and changes as fall within the true spirit of the invention.

What is claimed:

1. A medical device comprising:
a pumping mechanism for administering a fluid to a patient, wherein said pumping mechanism is characterized by having two or more treatment routes by which to connect to a patient;
a controller for controlling the pumping mechanism;
one or more inputs for receiving information including an intended route indication; and
a display to visually label the medical device according to the selected route indication.

2. The medical device according to claim 1, wherein said intended route indication is selected from the group consisting of: intra-venous (IV), subcutaneous, epidural, enteral, intrathecal, subarachnoid, peri-neural and neuro-axial.

3. The medical device according to claim 1, wherein said pump mechanism for administering fluid is selected from the group consisting of: saline, water, total parenteral nutrition (TPN), lipids, IV medication, epidural medication, blood and blood products.

4. The medical device according to claim 1, further comprising a memory storing a drug library including permissible route information.

5. The medical device according to claim 4, wherein said controller is configured to compare said intended route indication with said permissible route information and disable said pumping mechanism if an unallowable combination is indicated.

6. The medical device according to claim 5, wherein said controller is configured to compare said intended route indication and disable said pumping mechanism if said actual treatment route and said intended treatment route are non-matched.

7. The medical device of claim 1, wherein said to visually label a medical device is selected from the list consisting of: coloring a screen background, enabling a LED emitting a specific color, and displaying a route name.

8. The medical device of claim 1, wherein said display is further configured to display a ward in which the medical device is operating.

9. A medical server comprising:
a drug library including permissible route field;
program setting to be relayed to a medical device including a medical device identification and an intended treatment; and
a transceiver to (a) periodically relay to one or more medical devices the drug library to be stored locally on the one or more medical devices and to (b) relay to a specific medical device of the one or more medical devices a program setting; wherein said specific medical device is configured to compare the program setting with the permissible routes of the stored drug library and to disable the medical device if they are non-matched.

10. A method of operating a medical device comprising:
selecting a treatment route;
correlating between the treatment route with one or more program parameters;
producing an indication indicating either a non-allowable combination if the treatment route is incompatible with said at least one or more parameters or an allowable combination; and
disabling said medical device if said indication is a non-allowable combination.

11. The method according to claim 10, further comprising visually displaying said treatment route.

12. The method according to claim 11 wherein said program parameters are selected from the list of parameters comprising: a fluid type, a delivery mode, a dose of the fluid, the time of the treatment, and an identification of the medical device.

13. The method according to claim 11, wherein said treatment route is selected from the group consisting of: intra-venous (IV), subcutaneous, epidural, enteral, intrathecal, subarachnoid, peri-neural and neuro-axial.

14. The method according to claim 11, further comprising receiving a drug library including at least one permissible routes for each entry in the drug library.

15. The method according to claim 10, comprising visually displaying a ward where the medical device is located.
